(19) United States
(12) Patent Application Publication
(10) Pub. No.: US 2012/0059223 A1
(43) Pub. Date: Mar. 8, 2012

(54) LARYNGSCOPE INSERTION SECTION WITH TUBE GUIDE

(76) Inventors: Matthew John Ross McGrath, Edinburgh (GB); Morgan James Walker, Edinburgh (GB)

(21) Appl. No.: 13/254,579

(22) PCT Filed: Mar. 3, 2010
(86) PCT No.: PCT/GB10/50379

§ 371 Date: Nov. 16, 2011

(30) Foreign Application Priority Data
Mar. 3, 2009 (GB) 0903612.0

(51) Int. Cl. A61B 1/267

(52) U.S. Cl. 600/185

(57) ABSTRACT

A laryngoscope insertion section includes an elongate member and a tube guide having a moveable guiding member, located transversely of the elongate member and moveable relative to adjacent elongate member to adjust the path of a retained endotracheal tube, thereby facilitating intubation.

### FIELD OF THE INVENTION

The present invention relates to the field of laryngoscope insertion sections having tube guides for detachably retaining and guiding endotracheal tubes during intubation.

### BACKGROUND TO THE INVENTION

Laryngoscopes are medical devices which are employed to introduce endotracheal tubes into patient's airways, for example, when a patient is being anaesthetized. Laryngoscopes comprise insertion sections, which are the part of a laryngoscope which extends towards and into a patient's oral cavity during intubation. Insertion sections may be removably attachable to a laryngoscope body, integral parts of laryngoscopes or themselves function as laryngoscopes. As well as an insertion section, laryngoscopes typically comprises a handle which is usually elongate and which may be arranged at an angle to the proximal end of the insertion section or generally parallel to the proximal end of the insertion section, or at any angle therebetween. Laryngoscopes further include a source of light and a number of known devices, referred to as video laryngoscopes, include imaging devices, for example integral video cameras or fibre-optic bundles for attachment to external video cameras, to enable a user to view the distal tip of an endotracheal tube as it is being introduced into a patient's larynx.

Traditional laryngoscope insertion sections, such as insertion sections known in the art as Miller, Macintosh or Wisconsin blades, function to lift a patient's tissue adjacent the epiglottis to enable a tube to be inserted into a patient's larynx and to enable the patient's larynx to be viewed during intubation. However, they do not guide tubes as such.

A number of designs are known which do include a tube guide. For example, WO 04/073510 (Gandarias) discloses a laryngoscope having a lateral tube guide extending along the majority of the length of the insertion section. A tube guide enables an endotracheal tube to be detachably retained by the insertion section while it is introduced into a patient's larynx. In principal, the provision of a tube guide may facilitate intubation by introducing the endotracheal tube into the oral cavity at the same time as the insertion section and by directing a tube towards the larynx. However, a difficulty with known tube guides is that when an insertion section is introduced into the correct position to expose the larynx, the tube guide may not be arranged to direct a tube into the larynx as it is advanced through the tube guide. Furthermore, tube guides increase the overall bulk of insertion sections.

It has been proposed to provide a hinge in an insertion section to enable to longitudinally adjacent sections to pivot relative to each other around a lateral axis. However, this does not significantly facilitate intubation using a tube guide which is integral to the insertion section, particularly in difficult cases (referred to in the art as Grade 3 or Grade 4 intubations) where the larynx is not readily exposed and visible.

Thus, the invention aims to provide laryngoscopes and laryngoscope insertion sections which facilitate the introduction of an endotracheal tube into a patient's larynx.

### SUMMARY OF THE INVENTION

According to a first aspect of the present invention there is provided a laryngoscope insertion section having a proximal end and a distal end for insertion into a patient's oral cavity, the insertion section comprising a tube guide for retaining an endotracheal tube and guiding a retained endotracheal tube towards a patient's larynx, wherein the insertion section comprises an elongate support member and the tube guide comprises a moveable tube guiding member which is positioned transversely of the elongate support member and moveable relative to the elongate support member adjacent the moveable tube guiding member.

By ‘positioned transversely of’ we include the possibility that the moveable tube guiding member is mounted transversely of the insertion section, spaced apart from the surface of the moveable tube guiding member or formed in a transverse (e.g. superior, inferior or lateral) surface of the insertion section.

The moveable tube guiding member may be positioned inferiorly or superiorly of the elongate support member (and thereby positioned transversely of the moveable tube guiding member). Within this specification and the appended claims, the inferior surface is the surface of an insertion section which faces the patient's tongue in use. The opposite surface is referred to as the superior surface. Words such as inferior, inferiorly, superior and superiorly are used in corresponding senses. A superior-inferior axis is a virtual axis extending parallel to the superior and inferior directions.

The moveable tube guiding member may be positioned laterally of the insertion section (and thereby positioned transversely of the insertion section). For example, it may extend from the adjacent insertion section generally orthogonally to both the length of the insertion section and the superior-inferior axis.

The insertion section has a distal end. The tube guide, including the tube guiding member, defines at least in part a distal tube path along which a detachably retained endotracheal tube which has been advanced sufficiently far in a distal direction extends from the distal end of the insertion section towards a patient's larynx during intubation. Typically also, the tube guide, including the tube guiding member, defines, at least in part, a proximal tube path along which a detachably retained endotracheal tube is guided along at least the majority of the length of the insertion section.

Typically, the distal tube path defined at least in part by the tube guide will be slightly different for endotracheal tubes of different configuration, for example, of different external diameter. Thus, the distal tube path, and proximal tube path where relevant, and typically specific to endotracheal tubes of predetermined configuration (e.g. predetermined external diameter).

The distal tube path, and proximal tube path where relevant, may also be defined in part by the actions of a user, for example, by the orientation at which a user feeds an endotracheal into the insertion section tube guide, or the orientation at which a user holds an endotracheal tube relative to the proximal end of the insertion section.

Preferably, the moveable tube guiding member is moveable relative to the elongate support member to change the distal tube path (and typically also the proximal tube path where relevant).

Preferably, the moveable tube guiding member is moveable relative to the elongate support member to thereby displace the distal tube path. Preferably, the moveable tube guiding member is moveable relative to the elongate support member to thereby change the orientation of the distal tube path relative to the insertion section. Thus, the path along which a retained endotracheal tube having a particular configuration is guided towards a patient's larynx in use, during intubation, can be changed by movement of the moveable tube guiding member relative to the elongate support member.

Preferably, the moveable tube guiding member is moveable relative to the elongate support member to thereby displace and/or change the orientation of the distal tube path relative to the insertion section, independently of advancement of the endotracheal tube. Thus, the path at which an endotracheal tube extends towards a patient's larynx in use can be adjusted before the endotracheal tube is advanced into a patient's larynx. This provides a user with additional control of the intubation procedure.

Preferably, the moveable tube guiding member is moveable relative to the elongate support member to thereby displace and/or change the orientation of the distal tube path without concomitantly detaching a retained endotracheal tube from the tube guide.

Preferably, the moveable tube guiding member is moveable relative to the elongate support member to adjust the distal tube path in a superior or inferior direction, either or both displacing the distal tube path in a superior or inferior direction, or changing the orientation of the distal tube path in a plane including the endotracheal tube and the superior-inferior axis. We have found that adjustment of the distal tube path in a superior or inferior direction is typically of most benefit during intubation. Nevertheless, the moveable tube guiding member may be moveable relative to the elongate support member to adjust the lateral position of the distal tube path.

Preferably, the insertion section extends distally of the moveable tube guiding member. Thus, a given movement of the moveable tube guiding member will typically have a greater effect on the position of the distal end of a retained endotracheal tube extending beyond the distal end of the insertion section and adjacent to a patient's larynx in use than would be the case if the moveable tube guiding member was located at the distal end of the insertion section.

The moveable tube guiding member may be moveable prior to insertion of an endotracheal tube. The moveable tube guiding member may be moveable by one or more electric motors.

The insertion section may comprise a plurality of moveable tube guiding members. Movement of some or all of the plurality of moveable tube guiding members may be linked to facilitate adjustment of either or both of the position and orientation of a retained endotracheal tube, as appropriate. For example, two or more moveable tube guiding members may be mechanically connected and thereby be moveable concurrently (for example, mechanically coupled to moved concurrently).

A first moveable tube guiding member may be provided on the proximal side of the elongate support member in a distal region of the insertion section. A first moveable tube guiding member may be provided on the superior side of the elongate support member such that the first moveable tube guiding member is the most distal location where a tube retained in the tube guide contacts any portion of the tube guide. The first moveable tube guiding member may be moveable with a component parallel to the superior-inferior axis to move the distal tip of a retained endotracheal tube parallel to the superior-inferior axis in use.

A second tube guiding member may be provided proximally of the first moveable tube guiding member and spaced apart from the first moveable tube guiding member so that movement of the first moveable tube guiding member causes a retained endotracheal tube to pivot around the second tube guiding member. Thus, the tip of a retained endotracheal tube may move in geared relationship to movement of the first moveable tube guiding member. Typically the insertion section is arranged so that, in use, the tip of retained endotracheal tube adjacent to a patient's larynx in use moves in geared relationship to the movement of the first moveable tube guiding member with a gearing ratio of greater than 1.0.

The second tube guiding member may be a second moveable tube guiding member, located proximally of the first moveable tube guiding member and moveable with a component parallel to the superior-inferior axis to adjust the position and/or orientation at which a retained endotracheal tube extends from the second moveable tube guiding member to the first moveable tube guiding member and thus the distal path section. Typically, the first and second moveable tube guiding members are moveable together and the movement of each contributes to adjustment of the distal path section such that the resulting adjustment of the distal path section is greater than would be the case if only the first moveable tube guiding member or only the second moveable tube guiding member moved.

The moveable tube guiding member may be mounted to the elongate support member by way of a pivot. The tube guiding member may be mounted on a pivoting member, wherein the pivot is located proximally of the moveable tube guiding member. The moveable tube guiding member may comprise a movement limiting formation (for example the periphery of an aperture in the moveable tube guiding member) which engages with a cooperating formation of the elongate support member to limit the range of movement of the moveable tube guiding member. The moveable tube guiding member may be mounted to the elongate support member by way of a universal joint, for example, a ball and socket joint.

Preferably, the moveable tube guiding member is moveable whilst the insertion section is located within a patient ready for an endotracheal tube to be advanced into the patient's larynx.

Preferably, the laryngoscope insertion section further comprises a manually operable control to enable a user to move the moveable tube guiding member relative to the elongate support member and thereby vary the either or both of the orientation and position of a guided endotracheal tube relative to the elongate support member.

The manually operable control is preferably located to be operable with a user's fingers whilst the insertion section is located within a patient's oral cavity and either or both the position and orientation of the endotracheal tube are adjusted. Thus, the manually operable control is typically located to extend (and potentially to be located entirely) proximally of a patient's teeth when the insertion section is fully introduced into a patient's oral cavity for intubation.

The manually operable control may communicate with the moveable tube guiding member by way of a pulley. The manually operable control may communicate with the moveable tube guiding member by way of a pneumatic or hydraulic conduit. Thus, the moveable tube guiding member may be part of, or connected to a pneumatic or hydraulic actuator operably connected to the manually operable by control by a said pneumatic or hydraulic conduit. The moveable tube guiding member may be moveable by an electronically controllable motor, which may be in electronic communication with the manually operable control, by wired or wireless means. The electronically controllable motor may be in electronic communication with a remote tube moving control, by wired or wireless means.

The manually operable control may be integral to the moveable tube guiding member. For example, the moveable tube guiding member may comprise a manually operable lever extending proximally from the moveable tube guiding member and functioning as the manually operable control. In this case, the moveable tube guiding member is preferably mounted to the elongate support member by a pivot. The manually operable control may be a portion of a moveable control member which is coupled to the moveable tube guiding member. For example, each of the lever and the moveable tube guiding member may be mounted on respective pivoting members, which respective pivoting member are connected by way of a pivot to rotate together as the lever is manually operated.

Where the insertion section comprises a plurality of moveable tube guiding members, the manually operable control may be operable to cause a plurality or all of the moveable tube guiding members to move concurrently. For example, where said first and second moveable tube guiding members are provided operation of the manually operable control may be operable to cause the second moveable tube guiding member to move in a superior or inferior direction and the first moveable tube guiding member to concurrently move in the opposite direction. It may be that the insertion section comprises a pivoting member, pivotally attached to elongate support member and wherein the pivoting member comprises formations functioning as the first and second moveable tube guiding members so that the first and second moveable tube guiding member move concurrently by rotation of the pivoting member around the pivot. The pivoting member may comprise a manually operable lever.

The moveable tube guiding member may be pivotable to adjust the location and/or orientation at which a retained endotracheal tube extends towards a patient's larynx in use by manual manipulation of the retained endotracheal tube, either proximal of a patient's teeth, or within a patient's oral cavity, adjacent the teeth. The moveable tube guiding member is preferably located within the distal half of the region of the insertion section which is located within the mouth of a patient of median size during intubation.

The moveable tube guiding member may be moveable between a first position in which an endotracheal tube of a first external diameter cannot be retained by and guided by the tube guide and a second position in which an endotracheal tube of the first external diameter can be retained by and guided by the tube guide. Thus, the moveable tube guiding member may be used to reliably guide endotracheal tubes of a wider range of external diameters than if the tube guide were fixed. Thus the moveable tube guiding member may be moveable such that the distance between a tube contacting portion of the moveable tube guiding member and the elongate support member, adjacent the moveable tube guiding member, is varied.

The insertion section may be integral to a laryngoscope. The insertion section may be demountably attachable to a laryngoscope. Preferably, the insertion section comprises an elongate cavity extending along part of the length of the insertion section to enable the insertion section to be demountably attached to an insertion section retaining member of a laryngoscope.

Preferably, the tube guide is a tube guide for removably retaining an endotracheal tube.

According to a second aspect of the present invention there is provided a laryngoscope insertion section having a proximal end and a distal end for insertion into a patient's oral cavity, the insertion section comprising a tube guide for retaining an endotracheal tube and guiding a retained endotracheal tube towards a patient's larynx, wherein the inferior surface of the insertion section comprises a distal patient contacting point and the tube guide comprises a tube guiding mechanism to adjust the position of the distal tip of a retained endotracheal tube relative to the distal patient contacting point while the insertion section is positioned within a patient's oral cavity for intubation.

The tube guiding member mechanism may comprise a moveable tube guiding member. The moveable tube guiding member may be positioned transversely of the insertion section. The distal tube contacting point is typically the distal tip of the insertion section.

Thus, a user can introduce the insertion section and use the inferior surface of the insertion section to lift the tissue adjacent the epiglottis and view the larynx. The user can then adjust the position of the distal tip of a retained endotracheal tube, whilst the insertion section is positioned within a patient's oral cavity for intubation.

Further optional features of the second aspect of the invention correspond to the optional features described above in relation to the first aspect of the invention.

According to a third aspect of the invention there is provided a laryngoscope having an insertion section retaining formation to demountably retain an insertion section according to the first or second aspect of the invention. The invention also extends to a laryngoscope comprising a handle and an insertion section according to the first or second aspect of the invention fixedly attached thereto.

The laryngoscope preferably comprises a light source. The elongate cavity may be operable to encompass the light source in use and the insertion section may comprise a translucent or transparent portion to enable light from the light source to be shone on a patient's larynx in use. Thus, the insertion section may function to protect the light source from contact with bodily fluids and/or air during use.

The light source may be a light generating device, for example a light emitting diode or a bulb. The light source may be a light emitting region of a light conduit operably connected to or connectable to a light generating device.

The laryngoscope preferably comprises an image collector. The elongate cavity may be adapted to encompass an image collector in use. The image collector may be a camera. The image collector may comprise a light collecting region of a light conduit and the light conduit may be operable to conduct light to a camera.

The insertion section retaining formation may comprise an elongate image collector support including the image collector and arranged to extend into the elongate cavity in use so as to collect images of a region including a patient's larynx during intubation.

The elongate image collector support may comprise a rigid strengthening element. For example, it may comprise an elongate rigid metal housing. The light source may also be mounted in or on the elongate image collector. However, the elongate image collector support may be flexible.

In embodiments where the moveable tube guiding member is moveable responsive to operation of a manually operable control, the manually operable control may be located proximally of a patient's teeth in use, for example adjacent or on the handle of the laryngoscope. The laryngoscope may comprise a pivoting member having the moveable tube guiding member thereon, wherein the moveable tube guiding member is mounted on the moveable tube guiding member and the pivoting member is pivotably attached to the laryngoscope handle.

In embodiments including an image collector for imaging the larynx during intubation, and at least one manual control for controlling movement of the moveable tube guiding member, the image collector, the insertion section, at least one manual control and the moveable tube guiding member may be formed and arranged so that movement of a said at least one manual control moves the tip of a retained endotracheal tube vertically up or down in the images collected by the image collector. The image collector, the insertion section, a second manual control and the moveable tube guiding member may be formed and arranged so that movement of the second manual control moves the tip of a retained endotracheal tube left or right in the images collected by the image collector. This facilitates easy adjustment of the location of a retained endotracheal tube relative to a patient's larynx during intubation.

Where the insertion section is an insertion section according to the second aspect of the invention, the laryngoscope may further comprise a flexible tube guiding member extending from the handle of the laryngoscope adjacent the insertion section, which flexible tube guiding member is flexible responsive to operation of a manual control, to adjust the position the distal tip of a retained endotracheal tube relative to the distal patient contacting point while the insertion section is positioned within a patient's oral cavity for intubation.

The flexible tube guiding member may comprise a bougie for introduction into the bore of an endotracheal tube. The flexible tube guiding member may be adapted to contact an exterior surface of an endotracheal tube, for example, the flexible tube guiding member may comprise a channel within which an endotracheal tube can be retained.

The flexible tube guiding member may comprise an actuator operable to flex the flexible tube guiding member responsive to operation of a manual control. The actuator may be an electric motor. The actuator may be a hydraulic or pneumatic actuator. The actuator may comprise a shape memory alloy (e.g. “muscle wire”).

### DESCRIPTION OF THE DRAWINGS

An example embodiment of the present invention will now be illustrated with reference to the following Figures in which:

FIG. 1 is a side view of a laryngoscope insertion section retaining an endotracheal tube having an external diameter at the upper end of an operating range of external diameters, with the movable tube guide member in a tube raising configuration;

FIG. 2 is a side view of the laryngoscope insertion section, without a retained endotracheal tube, with the movable tube guiding member in a tube raising configuration;

FIG. 3 is a side view of the laryngoscope insertion section retaining an endotracheal tube having an external diameter at the upper end of the operating range of external diameters, with the movable tube guiding member in a tube lowering configuration;

FIG. 4 is a side view of the laryngoscope insertion section, without a retained endotracheal tube, with the movable tube guiding member in a tube lowering configuration;

FIG. 5 is a side view of the laryngoscope insertion section retaining an endotracheal tube having an external diameter at the lower end of the operating range of external diameters, with the movable tube guiding member in a tube raising configuration;

FIG. 6 is a cross-section through a patient, illustrating the position of the laryngoscope insertion section and an endotracheal tube having an external diameter at the lower end of the operating range of external diameters, when the movable tube guiding member is in the tube raising configuration, or in the tube lowering configuration;

FIG. 7 is a side view of the laryngoscope insertion section retaining an endotracheal tube having an external diameter at the low end of the operating range of external diameters, illustrating the position of the endotracheal tube with the movable tube guiding member in a tube raising configuration, or a tube lowering configuration;

FIG. 8 corresponds to FIG. 7, except that the illustrated endotracheal tube is an endotracheal tube having an external diameter at the lower end of the operating range of external diameters;

FIG. 9 is an isometric view of the distal tip of an insertion section according to FIG. 1 and a retained endotracheal tube;

FIG. 10 is an isometric view of the proximal end of an insertion section according to FIG. 1 and a retained endotracheal tube;

FIG. 11 is an isometric view of a laryngoscope without an attached insertion section;

FIG. 12 is an isometric view of the laryngoscope of FIG. 11 with an insertion section of FIG. 1 attached thereto;

FIG. 13 is a side view of an alternative embodiment of an insertion section, having to pivoting tube guide portions;

FIG. 14 is an isometric view of the insertion section of FIG. 13;

FIG. 15 is a plan view of the insertion section of FIG. 13;

FIG. 16 is a side view of a further alternative embodiment of an insertion section;

FIG. 17 is an isometric view of the insertion section of FIG. 16;

FIG. 18 is a plan view of the insertion section of FIG. 1, retaining an endotracheal tube at an upper end of the operating range of endotracheal tube external diameters; and

FIG. 19 is a detail of the distal tip of the insertion section and retained tube of FIG. 18.

FIGS. 20a and 20b illustrate a laryngoscope having an insertion section and a bougie extending from the laryngoscope handle adjacent the insertion section and operable responsive to a manual control;

FIGS. 21a and 21b illustrate a laryngoscope in which the moveable tube guide is operable by a manual control located on the laryngoscope handle.

### DETAILED DESCRIPTION OF AN EXAMPLE EMBODIMENT

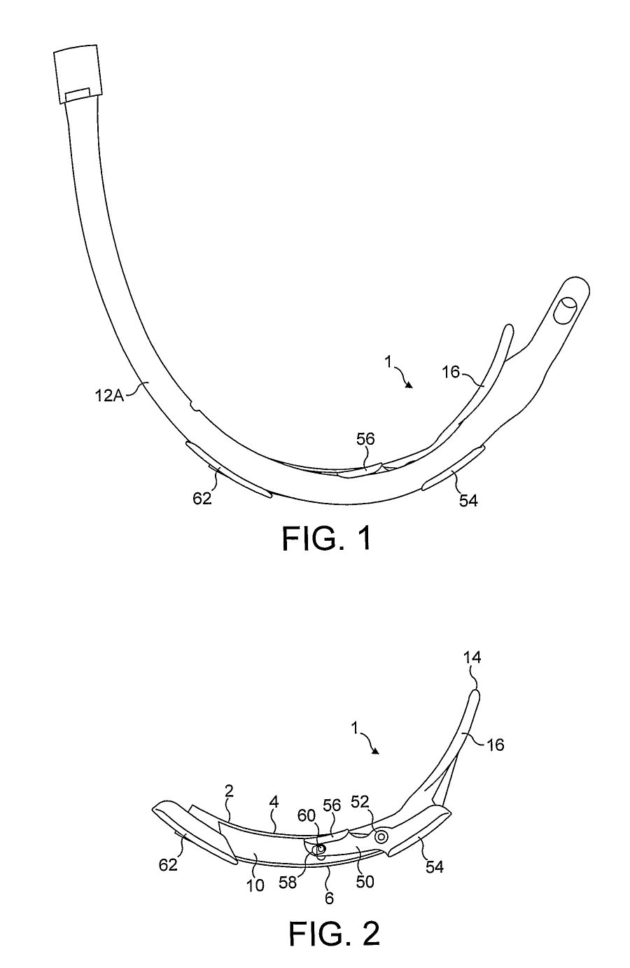

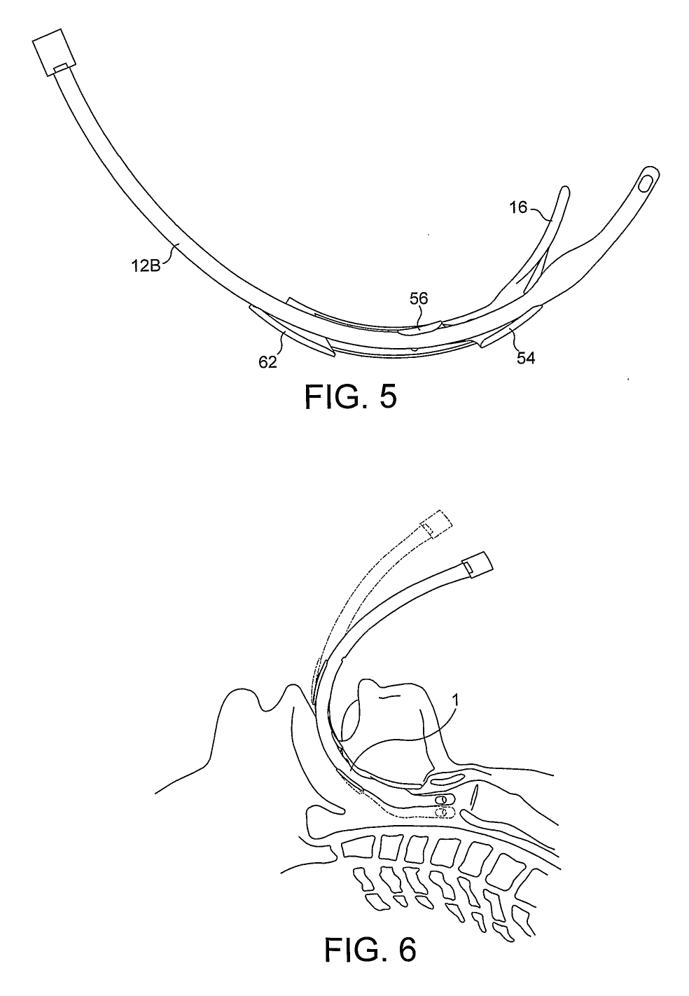

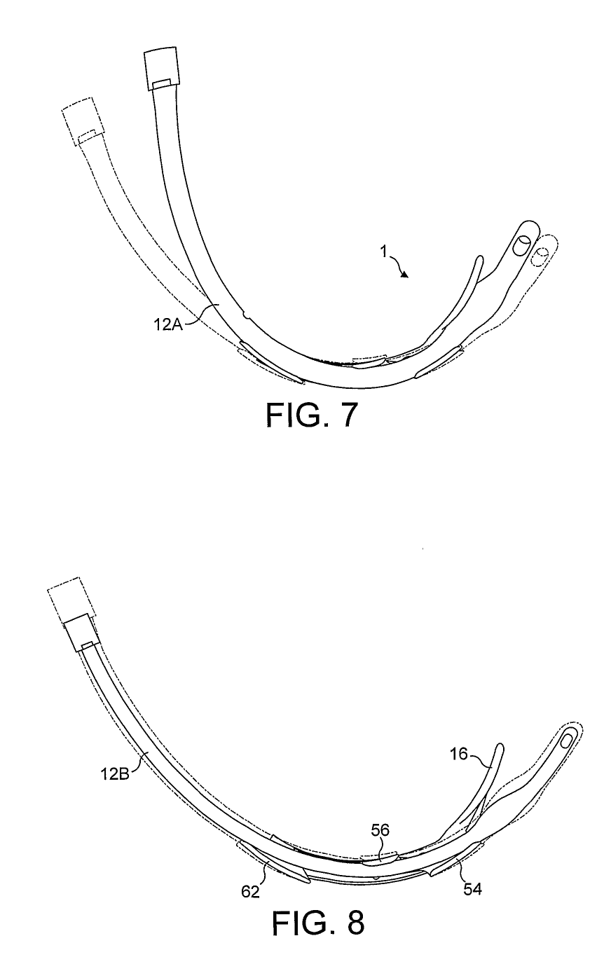

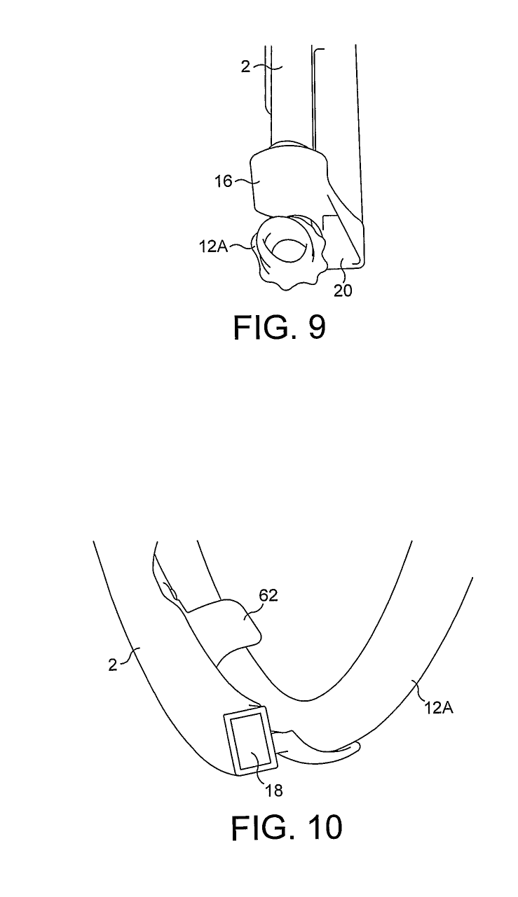

With reference to FIGS. 1 through 8, a laryngoscope insertion section, shown generally as 1, has a body 2, formed as a unitary moulding from a transparent plastics material and functioning as the elongate support member. The body has a smooth inferior surface 4, which contacts a patient's palette in use, an opposing superior surface 6, a first smooth lateral surface 8, and an opposing second lateral surface 10. The second lateral surface has a profile including a concave groove which runs along the majority of the length of the second lateral surface and which functions as part of a tube guide for an endotracheal tube 12A, 12B. The insertion section has a distal end 14 comprising a spatula member 16 which functions, in use, to lift a patient's anatomy adjacent the epiglottis.

The insertion section further defines an elongate cavity 16 which extends along a part of the length of the insertion section from an aperture 18. The elongate cavity is closed off at its distal end by a window 20. The elongate cavity curves gently and is sized to retain a support member 104 of a laryngoscope 100, illustrated in FIG. 10. The distal end of the elongate cavity is sealed to protect a camera 106 provided at the distal end of the laryngoscope support member from bodily fluids in use, while providing a viewing port through which the camera can image a patient's larynx during intubation. The insertion section is formed and arranged to fit over the support member like a sleeve. The insertion section is elongate and curved, and generally within a plane.

A pivoting member 50 is attached to the second lateral surface of the insertion section by way of a pivot 52. The pivoting member includes a distal superior tube guiding member 54 (functioning as the moveable tube guiding member), located distally of the pivot, having a concave elongate groove on an inferior surface thereof, the distal tip of which contacts the superior surface of a retained endotracheal tube in use. The pivoting member includes an inferior tube guiding member 56 (functioning as a further moveable tube guiding member), located proximally of the pivot, having a concave elongate groove on a superior surface thereof, at least the proximal tip of which contacts the inferior surface of a retained endotracheal tube in use. The pivoting member is rotatable around the pivot, in the plane of the insertion section, and its movement is limited by the periphery of an aperture 58 through the pivoting member, which engages with a pin 60 extending from the second lateral surface of the insertion section. As the pivoting member can be rotated in use, the position of the distal superior tube guiding member, which is positioned transversely of (in this example mounted transversely to) the body of the insertion section, can be moved relative to the body of the insertion section adjacent the distal superior tube guiding member.

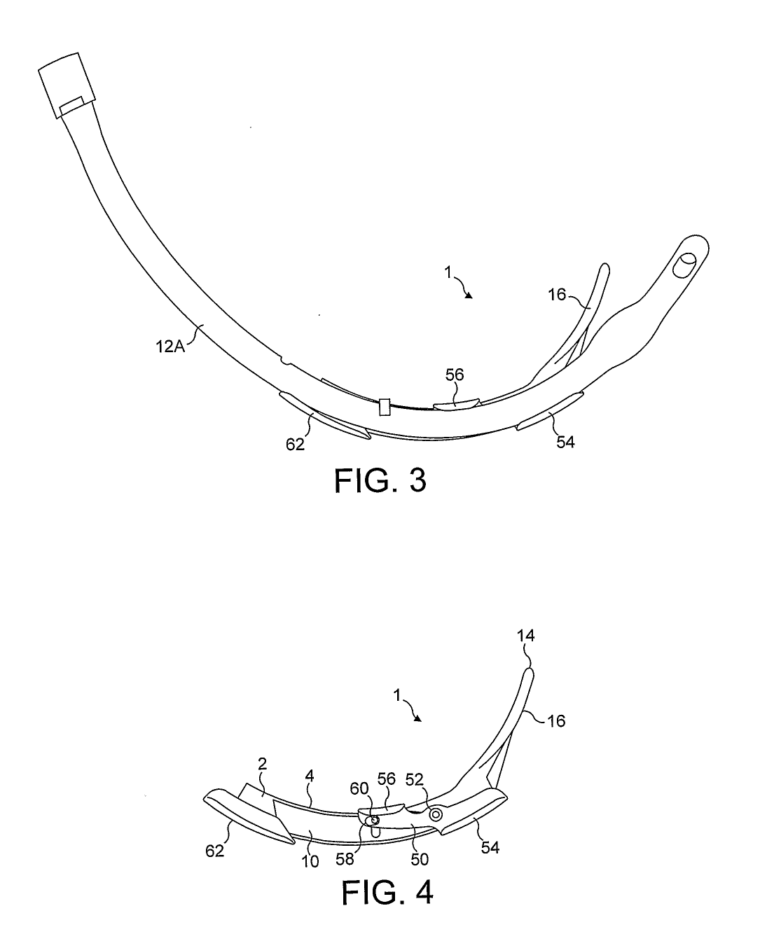

A tube guide is formed by the distal superior tube guiding member and the inferior tube guiding members, as well as a proximal superior tube guiding member 62, which, in this embodiment, is fixedly mounted to the insertion section, located towards the proximal end of the insertion section, having a concave elongate groove on an inferior surface thereof, at least the distal tip of which contacts the superior surface of a retained endotracheal tube in use. The concave elongate groove of the proximal superior tube guiding member extends slightly in the superior direction towards its distal tip, to retain an endotracheal tube at a smaller radius of curvature than the radius of curvature of the insertion section at the distal to the third tube guiding member, as can be seen from FIG. 1 and FIG. 3. The proximal tip of the inferior tube guiding member is longitudinally spaced from the distal tip of the proximal superior tube guiding member so that the endotracheal tube can follow a path with a smaller radius of curvature than the insertion section. This arrangement enables the tube guide to be especially thin in the region of a patient's teeth and, as the inferior surface of a retained endotracheal tube is left exposed, the endotracheal tube may be grasped by the user. As will be evident from FIG. 1 and FIG. 3, the insertion section is also useful with endotracheal tubes of a wide range of external diameters, which follow slightly different paths along the length of the insertion section.

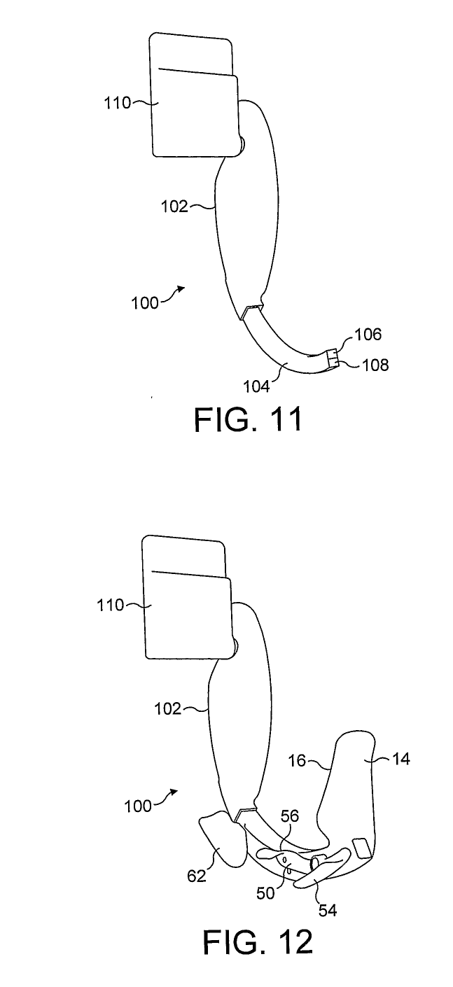

The insertion section is for use as a disposable accessory for a laryngoscope 100, illustrated in FIGS. 11 and 12. The laryngoscope includes a handle 102 from which support member 104 extends. The support member has, at a distal tip, camera 106 (functioning as a light collector) and an LED light source 108. The support member is formed from steel and provides mechanical support for an insertion section retained on the support member in use. A battery within the handle (not shown) provides power to the camera and light source. A video screen 110 receives and displays images from the camera in use. The support member may be integral to the body of the laryngoscope or demountable, for example, to enable the support member to be separately sterilised. The junction between the laryngoscope body and support member may be adjustable to vary the maximum distance to which the support member extends from the laryngoscope body. The support member curves gently within a plane. The plane of the support member is optionally laterally offset from the central axis of the laryngoscope.

In use, a new disposable insertion section, which has typically been kept in a sterile package since manufacture, is slid onto the retaining member until the tip of the retaining member is adjacent the transparent window. The insertion section protects the retaining member and the camera and light source at the distal end of the retaining member. Furthermore, light from the light source is directed towards and beyond the distal tip of the insertion section and the camera is operable to collect images of the distal tip of the insertion section and the surrounding space. As the retaining member is formed from steel, it functions as a strengthening element, reinforcing the insertion section.

Next, an endotracheal tube is inserted into the tube guide. The endotracheal tube is held in flexural tension by the proximal and distal superior tube guiding member and the inferior tube guiding member. The proximal and distal superior tube guiding members exert forces in an inferior direction on the endotracheal tube and the inferior tube guiding member exerts forces in a superior direction, such that the endotracheal tube is both held in placed and guided along a path. The flexural tension serves to retain the tube in position and avoids the requirement for further tube retaining members. Furthermore, the position of the proximal and distal superior tube guiding member and the inferior tube guiding member defines the path by which a retained endotracheal tube having a given external diameter extends along the insertion section (functioning as the proximal tube path) when no external force is applied to the endotracheal tube. The position of the proximal and distal superior tube guiding members and the inferior tube guiding member further defines the path (the distal tube path) by which a retained endotracheal tube having a given external diameter would extend beyond the insertion section, towards a patient's larynx in use, when no external force is applied to the endotracheal tube.

Initially, a retained endotracheal tube extends distally of the distal superior tube guiding member but is preferably not sufficiently far advanced as to extend beyond the distal tip of the insertion section. The endotracheal tube is retained in flexural tension from the most proximal location where it contacts the tube guide to the most distal location where it contacts the tube guide. As a result of this flexural tension, and the gentle curvature which is typical of endotracheal tubes, the tube exerts a force in the superior direction on the distal superior tube guiding member, as well as a force in the inferior direction on the inferior tube guiding member. Thus, the pivoting member will typically rest in a position determined by the movement limiter, referred to as the tube lowering position and illustrated, for example, in FIG. 3.

The laryngoscope is then manipulated to introduce the insertion section into a patient's oral cavity, using the spatulate tip portion to lift the patient's anatomy around the epiglottis, exposing the larynx. During this stage, images from the camera are transmitted by a wired or wireless connection to a display screen which can conveniently be attached to the handle of the laryngoscope but may alternatively be separate to the laryngoscope. As the endotracheal tube is already retained within the tube guide, the user will not be required to carry out the additional step of introducing an endotracheal tube into a patient's oral cavity, freeing up one of their hands. However, the laryngoscope, insertion section and tube guide may also be configured so that an endotracheal tube can be introduced into the tube guide and advanced along the tube guide after insertion of the laryngoscope into a patient's oral cavity.

The user will adjust the position of the laryngoscope so as to provide a good view of the patient's larynx. Typically, the endotracheal tube will not initially be aligned perfectly to advance into the larynx. The insertion section is designed so that when the distal tip of the insertion section is in the correct position to best lift the patient's anatomy around the epiglottis and expose the larynx, an endotracheal tube located in the tube guide will typically be located at, or close to, the correct lateral position to advance a tube into a patient's larynx. However, there may well be a significant difference between the position of the distal tip of the endotracheal tube parallel to the inferior-superior axis, or the angle at which the distal tip of the endotracheal tube extends in the plane of the insertion section. (The plane of the insertion section is typically a vertical plane in use as patients are virtually always intubated lying on their backs with their midsagittal plane vertical, and their neck tilted backwards).

The user then adjusts the orientation of the distal tip of the endotracheal tube in the plane of the insertion section by contacting the endotracheal tube with their fingers adjacent to and possibly distally of the proximal superior tube guiding member which, when the larynx is fully in view, is typically adjacent to or just outside of a patient's teeth. As users of laryngoscopes typically grip laryngoscopes at the base of the handle and proximal end of the insertion section, this is a natural movement. By slight movements of their fingers, users can change the orientation at which the endotracheal tube contacts the distal end of the proximal superior tube guiding member. By increasing the angle of incidence of the endotracheal tube on the distal end of the proximal superior tube guiding member, the pivoting member pivots such that the inferior tube guiding member moves in an superior direction and the distal superior tube guiding member moves in an inferior direction. As a result, the tip of the retained endotracheal tube moves in an inferior direction. As the inferior tube guiding member and distal superior tube guiding member are linked and move together, and as the insertion section extends distally of the distal superior tube guiding member, a relatively small adjustment of the angle of incidence of the endotracheal tube on the distal end of the proximal superior tube guiding member can have a substantial effect on the movement of the distal tip of the endotracheal tube. Movement of the distal tip of the endotracheal tube in an inferior direction is effectively geared to movement of the distal superior tube guiding member in an inferior direction, with a gearing ratio of greater than 1.0 so that a compact insertion section can readily control significant movements in the distal tip of the retained endotracheal tube. The camera and display screen are typically configured so that the inferior direction is uppermost and so manually increasing the angle of incidence of the endotracheal tube will appear to move the tip of the endotracheal tube upwards on the display. The maximum extent to which the distal tip of the retained endotracheal tube can be moved in the inferior direction, referred to here as the tube raising position, is illustrated, for example, in FIG. 1.

Once the user is happy with the location of the distal tip of the endotracheal tube relative to the larynx, the user can advance the tube with their fingers, thereby intubating the patient. Advantageously the user has been able to locate the spatulate tip of the laryngoscope insertion section while concentrating on lifting the tissue adjacent the epiglottis to best expose the larynx, without having to adjust the location of the insertion section to orient the retained endotracheal tube relative to the patient's larynx. They can subsequently focus their attention on positioning the distal tip of the endotracheal tube, using their fingers, to direct the endotracheal tube towards a patient's larynx. As movement of the tip of the endotracheal tube in the inferior or superior direction can be accomplished without significantly advancing the endotracheal tube, the endotracheal tube can be advanced as a separate action. Finally, the endotracheal tube is detached from the tube guide and the laryngoscope and attached insertion section are removed from the oral cavity, leaving the endotracheal tube. The insertion section can then be disposed of and the laryngoscope reused for further intubations.

Typically, the insertion section is designed to be used with endotracheal tubes of a range of sizes. The range of external diameters of endotracheal tubes with which an insertion section can be reliably used is referred to as the an operating range of endotracheal tube sizes. The operating range of endotracheal tube size, and the dimensions of the insertion section will depend on the application of the insertion section. An insertion section for use with adult humans may, for example, be adapted to be usable reliably with endotracheal tubes with an external diameter of up to 12.3 mm. Tubes of this external diameter are referred to as Size 9.0 in the field. The minimum external diameter may be around 5.5 mm. Where the insertion section is made from a plastics material, the mean thickness of the inferior and first superior tube guiding members typically requires to be at least 0.75 mm (preferably around 1.5 mm) to provide suitable mechanical strength for internal use. Accordingly, the thickness of the first region is preferably less than 15.3 mm, more preferably less than 14.6 mm, 13.8 mm or more preferably less than 13.1 mm, in the case of an insertion section for inserting endotracheal tubes into adult humans.

The dimensions of an insertion section for use with infant humans, including new born infants, are typically scaled proportionately from the dimensions of an insertion section for use with human adults. Nevertheless, the proportions of some features, such as the thickness of the tube guiding members, may not scale proportionately. In the case of an insertion section for inserting endotracheal tubes into infant humans, including new born infants, the operating range of external tube diameters may be 1.0 to 5.0 mm, and the thickness of the first region is preferably less than 8.0 mm, preferably less than 7.0 mm, or more preferably less than 6.0 mm.

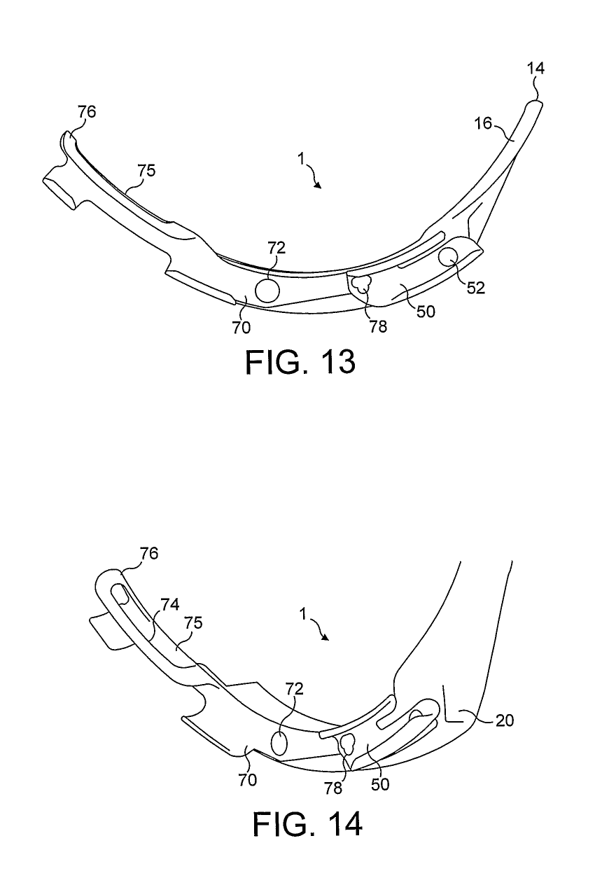

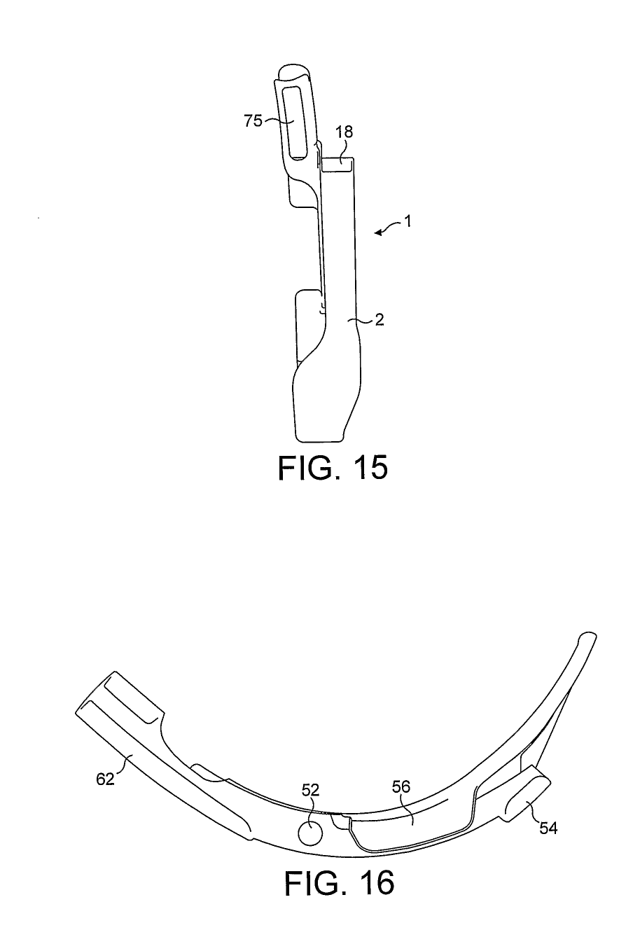

FIG. 13 through 15 illustrate an alternative embodiment of an insertion section, which includes a pivoting member 50 and a control member 70 which is also rotatable, around a pivot 72. The control member includes a lever 74, proximal superior tube guiding member and proximal inferior tube guiding member 76, having an elongate aperture 75 in the inferior face thereof to enable a user to contact and advance a retained endotracheal tube. The tube guiding member is attached to the pivoting member by way of a further pivoting joint 78. As a result of this arrangement, the control member and pivoting member rotate together. Thus, a user can adjust the position of the distal superior tube guiding member and therefore the position of the distal tip of a retained endotracheal tube, by manual adjustment of the angle between the lever and the insertion section. This angle may be adjusted using by directly rotating the lever, or by holding and adjusting the trajectory of the endotracheal tube.

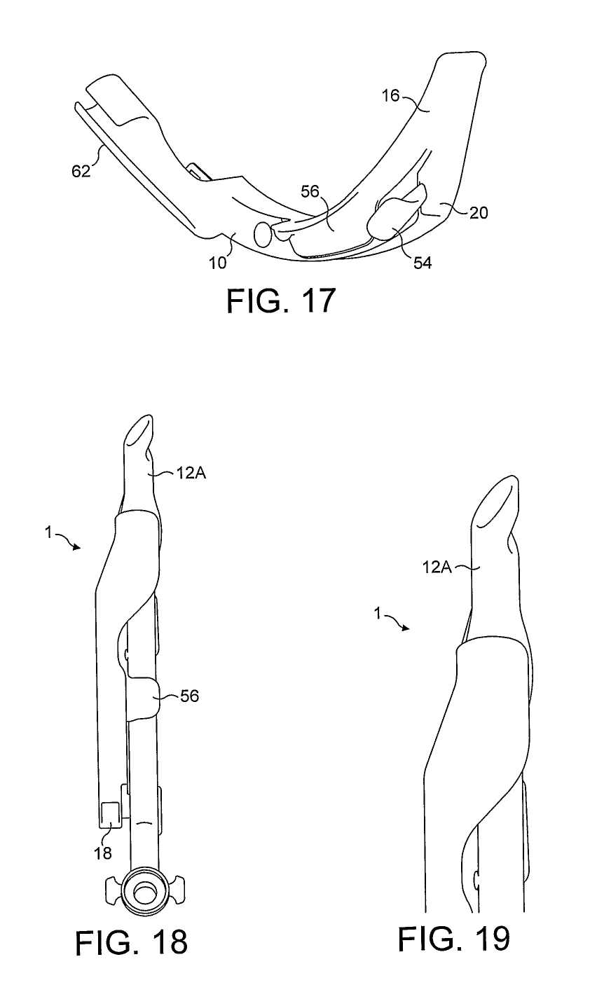

FIGS. 16 and 17 illustrate a further embodiment of an insertion section in which the pivoting member has an integral lever 80 so that the position of the distal superior tube guiding member in the superior-inferior direction and therefore the location of the distal tip of a retained endotracheal tube can be adjusted by varying the angle of the integral lever relative to the adjacent insertion section.

One or more moveable tube guiding members can be moved relative to the adjacent insertion section body by other means. For example, an electronic motor may be employed to move the distal superior tube guiding member, and optionally one or more other tube guiding members, in an inferior or superior direction, to thereby adjust the path at which a retained endotracheal tube extends toward a patient's larynx in use. The electric motor can be controlled by manually operable controls (such as a dial or buttons) on the laryngoscope handle, insertion section, or remotely, and control signals can be transmitted to the electric motor by wired or wireless connection. Two or more tube guiding members may move concurrently, in the same or opposite senses, under the control of a single motor.

A tube guiding member can also be moved relative to adjacent insertion section by a pneumatic or hydraulic actuator connected to a manually operable control (such as a button) located on the laryngoscope handle or insertion section by a pneumatic or hydraulic conduit. The pneumatic or hydraulic actuator may be a balloon. The tube guiding member may be, of be formed on, a surface of a pneumatic or hydraulic actuator which inflates or deflates responsive to operation of a manually operable control.

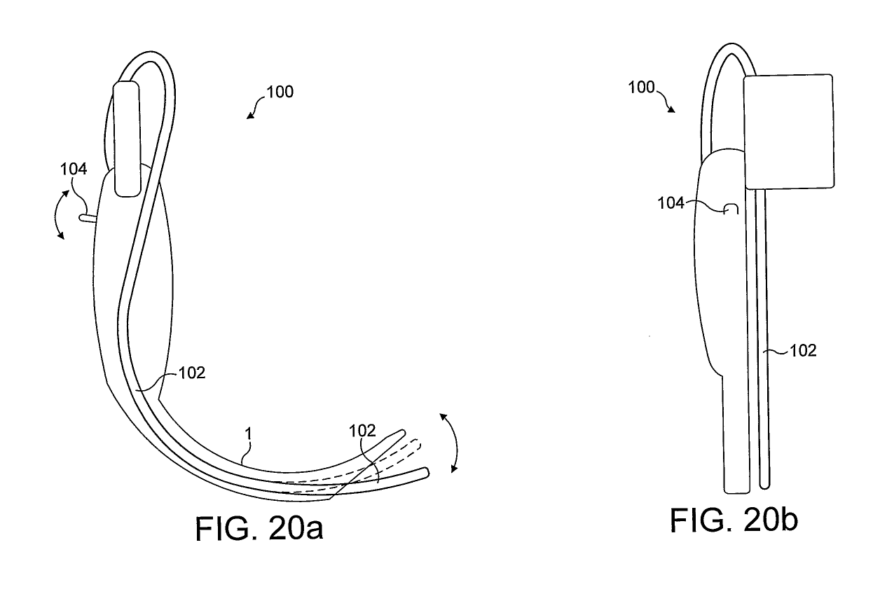

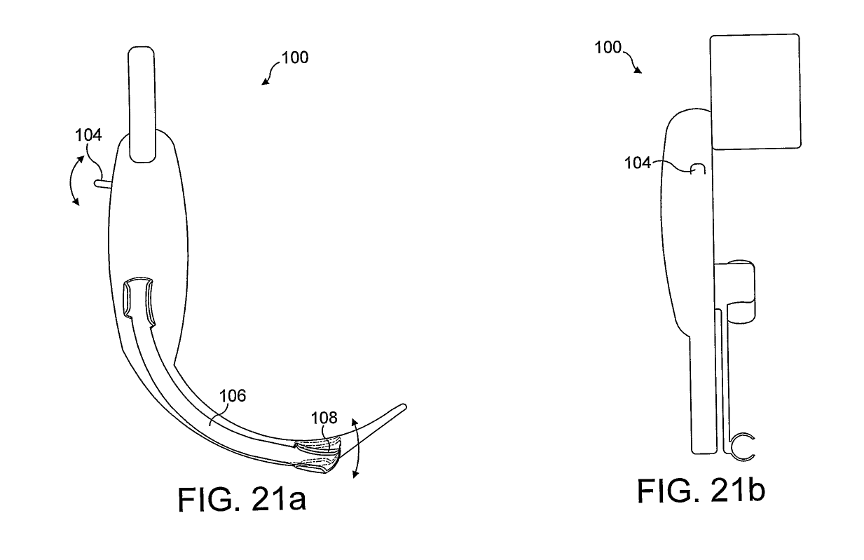

FIGS. 20a and 20b illustrate a video laryngoscope 100 having an insertion section 1 and a bougie 102 extending from the laryngoscope handle adjacent the insertion section and operable responsive to a manual control 104. Movement of the manual control up and down leads to a corresponding or opposite movement of the distal tip of the bougie. FIGS. 21a and 21b illustrate a video laryngoscope 100 having a moveable tube guide 106 operable by a manual control 104 located on the laryngoscope handle. Movement of the manual control up and down leads to a corresponding or opposite movement of a distal tube contacting portion 108 of the tube guide and a corresponding movement of the distal tip of an endotracheal tube retained in the tube guide.

Further variation and modifications may be considered by one skilled in the art, within the scope of the invention herein disclosed.

1. A laryngoscope insertion section having a proximal end and a distal end for insertion into a patient's oral cavity, the insertion section comprising a tube guide for retaining an endotracheal tube and guiding a retained endotracheal tube towards a patient's larynx, wherein the insertion section comprises an elongate support member and the tube guide comprises a moveable tube guiding member which is positioned transversely of the elongate support member and moveable relative to the elongate support member adjacent the moveable tube guiding member.

2. A laryngoscope insertion section according to claim 1, wherein the moveable tube guiding member is positioned laterally of the insertion section.

3. A laryngoscope insertion section according to claim 1, wherein the tube guide, including the tube guiding member, defines at least in part a distal tube path along which a detachably retained endotracheal tube which has been advanced sufficiently far in a distal direction extends from the distal end of the insertion section towards a patient's larynx during intubation, wherein the moveable tube guiding member is moveable relative to the elongate support member to change the distal tube path.

4. A laryngoscope insertion section according to claim 3, wherein the moveable tube guiding member is moveable relative to the elongate support member to thereby displace the distal tube path.

5. A laryngoscope insertion section according to claim 3, wherein the moveable tube guiding member is moveable relative to the elongate support member to thereby change the orientation of the distal tube path relative to the insertion section.

6. A laryngoscope insertion section according to claim 3, wherein the moveable tube guiding member is moveable relative to the elongate support member to thereby displace and/or change the orientation of the distal tube path relative to the insertion section, independently of advancement of the endotracheal tube.

7. A laryngoscope insertion section according to claim 3, wherein the moveable tube guiding member is moveable relative to the elongate support member to thereby displace and/or change the orientation of the distal tube path without concomitantly detaching a retained endotracheal tube from the tube guide.

8. A laryngoscope insertion section according to claim 3, wherein the moveable tube guiding member is moveable relative to the elongate support member to adjust the distal tube path in a superior or inferior direction, either or both displacing the distal tube path in a superior or inferior direction, or changing the orientation of the distal tube path in a plane including the endotracheal tube and the superior-inferior axis.

9. A laryngoscope insertion section according to claim 1, wherein the insertion section extends distally of the moveable tube guiding member.

10. A laryngoscope insertion section according to claim 1, wherein the insertion section comprises a plurality of moveable tube guiding members, wherein movement of some or all of the plurality of moveable tube guiding member is linked to facilitate adjustment of either or both of the position and orientation of a retained endotracheal tube.

11. A laryngoscope insertion section according to claim 10, wherein two or more moveable tube guiding members are mechanically connected and thereby moveable concurrently.

12. A laryngoscope insertion section according to claim 10, wherein the first moveable tube guiding member is provided on the proximal side of the elongate support member in a distal region of the insertion section, such that the first moveable tube guiding member is the most distal location where a tube retained in the tube guide contacts any portion of the tube guide, the first moveable tube guiding member is moveable with a component parallel to the superior-inferior axis to move the distal tip of a retained endotracheal tube parallel to the superior-inferior axis in use, and a second tube guiding member is provided proximally of the first moveable tube guiding member and spaced apart from the first moveable tube guiding member so that movement of the first moveable tube guiding member causes a retained endotracheal tube to pivot around the second tube guiding member.

13. A laryngoscope insertion section according to claim 1, wherein the moveable tube guiding member is mounted to the elongate support member by way of a pivot.

14. A laryngoscope insertion section according to claim 1, wherein the laryngoscope insertion section further comprises a manually operable control to enable a user to move the moveable tube guiding member relative to the elongate support member and thereby vary the either or both of the orientation and position of a guided endotracheal tube relative to the elongate support member.

15. A laryngoscope insertion section according to claim 14, wherein the manually operable control is integral to the moveable tube guiding member.

16. A laryngoscope insertion section according to claim 14, wherein the manually operable control is a portion of a moveable control member which is coupled to the moveable tube guiding member.

17. A laryngoscope insertion section according to claim 14, wherein the insertion section comprises a plurality of moveable tube guiding members, and wherein the manually operable control is operable to cause a plurality or all of the moveable tube guiding members to move concurrently.

18. A laryngoscope insertion section according to claim 1, wherein the moveable tube guiding member is pivotable to adjust the location and/or orientation at which a retained endotracheal tube extends towards a patient's larynx in use by manual manipulation of the retained endotracheal tube, either proximal of a patient's teeth, or within a patient's oral cavity, adjacent the teeth.

19. A laryngoscope insertion section according to claim 1, wherein the moveable tube guiding member is moveable between a first position in which an endotracheal tube of a first external diameter cannot be retained by and guided by the tube guide and a second position in which an endotracheal tube of the first external diameter can be retained by and guided by the tube guide.

20. A laryngoscope having an insertion section retaining formation to demountably retain an insertion section according to claim 1.

21. A laryngoscope comprising a handle and an insertion section according to claim 1 fixedly attached thereto.

22. A laryngoscope insertion section having a proximal end and a distal end for insertion into a patient's oral cavity, the insertion section comprising a tube guide for retaining an endotracheal tube and guiding a retained endotracheal tube towards a patient's larynx, wherein the inferior surface of the insertion section comprises a distal patient contacting point and the tube guide comprises a tube guiding mechanism to adjust the position of the distal tip of a retained endotracheal tube relative to the distal patient contacting point while the insertion section is positioned within a patient's oral cavity for intubation.

23. A laryngoscope insertion section according to claim 22, wherein the distal tube contacting point is the distal tip of the insertion section.

24. A laryngoscope having an insertion section retaining formation to demountably retain an insertion section according to claim 22.

25. A laryngoscope comprising a handle and an insertion section according to claim 22 fixedly attached thereto.

26. A laryngoscope according to claim 24, wherein the laryngoscope further comprises a flexible tube guiding member extending from the handle of the laryngoscope adjacent the insertion section, which flexible tube guiding member is flexible responsive to operation of a manual control, to adjust the position the distal tip of a retained endotracheal tube relative to the distal patient contacting point while the insertion section is positioned within a patient's oral cavity for intubation.

27. A laryngoscope according to claim 26, wherein the flexible tube guiding member comprises a bougie for introduction into the bore of an endotracheal tube.

28. A laryngoscope according to claim 26, wherein the flexible tube guiding member comprises a channel within which an endotracheal tube can be retained.

29. A laryngoscope according to claim 26, wherein the flexible tube guiding member comprises an actuator operable to flex the flexible tube guiding member responsive to operation of a manual control.

30. A laryngoscope according to claim 29, wherein the actuator is selected from a group comprising: an electric motor, a hydraulic actuator, a pneumatic actuator, an actuator comprising a shape memory alloy.
